(19) United States
(12) Patent Application Publication
(10) Pub. No.: US 2014/0228329 A1
(43) Pub. Date: Aug. 14, 2014

(54) USE OF 25-HYDROXY VITAMIN D3 TO PROMOTE PHOSPHOROUS UTILISATION IN RUMINANTS

(71) Applicant: Bob Elliott, Basel (CH)

(72) Inventor: Bob Elliott, Basel (CH)

(73) Assignee: DSM IP ASSETS B.V., Heerlen (NL)

(21) Appl. No.: 14/122,445

(22) PCT Filed: May 29, 2012
(86) PCT No.: PCT/EP2012/059996

§ 371 Date: Apr. 17, 2014

(30) Foreign Application Priority Data
May 26, 2011 (EP) 11167576.5

(51) Int. Cl. A61K 31/593; A23K 1/16

(52) U.S. Cl. CPC A61K 31/593; A23K 1/1603
USPC 514/167

(57) ABSTRACT

It has been found that administering 25-hydroxy vitamin D3 to ruminants helps to eliminate or at least reduce the need for supplemental inorganic P in the diets while maintaining milk production or growth performance. Furthermore, the excretion of P with the manure will be reduced and thus a positive effect on the environment will be achieved. Thus one aspect of this invention is a method of promoting phosphorus utilization in ruminants, comprising administering a sufficient amount of 25-hydroxy vitamin D3 to the animal. Another aspect of the invention relates to the use of a 25-hydroxy vitamin D3 for promoting or improving phosphorous utilization in ruminants and for reducing the excretion of P via faeces and urine.

It is well recognized that animals respond to Phosphorous (P) supplementation in terms of improved breeder fertility, improved frame size and growth rates. Large areas of grazing land around the world in particular Sth Africa, Sth America and Northern Australia are extremely Phosphorus deficient. Supplementation with Phosphorus is commonly used to improve animal productivity in these areas.

A large amount of phosphate is also present in animal feed in form of phytate phosphorus. In particular, the alkali or earth alkali salts of phytic acid occur naturally mainly in grains and cereals.

Phosphorous present in the diet not absorbed and not incorporated into tissue or milk is excreted via faeces and urine. The presence of P in manure has serious environmental consequences, and some countries have enacted strict legal controls on the amount of P which can be present in manure.

It is the object of the present invention to suggest a method of promoting or improving phosphorous utilization in ruminants which also reduces the level of phosphorous pollution of the environment

In accordance with the present invention it has been found that administering 25-hydroxy vitamin D3 to ruminants helps to increase Phosphorus absorption and maintain higher blood Phosphorus concentrations in cattle offered a low Phosphorus diet. This may also eliminate or at least to reduce the need for supplemental inorganic P in the diets while maintaining milk production or growth performance. Furthermore, the excretion of P with the manure will be reduced and thus a positive effect on the environment will be achieved.

Thus one aspect of this invention is a method of promoting phosphorus utilization in ruminants, comprising administering a sufficient amount of 25-hydroxy vitamin D3 to the animal.

Another aspect of the invention relates to the use of a 25-hydroxy vitamin D3 for improving phosphorous utilization in ruminants and for reducing the excretion of P via faeces and urine.

As used throughout the specification and claims, “25-OH D3” means 25-hydroxy vitamin D3.

There are about 150 species of ruminants which include both domestic and wild species. Ruminants include cattle (bovines), goats, sheep and camels. Preferred ruminants according to this invention are cattle, for example dairy cows and beef cattle.

While many benefits of supplementing a diet with Vitamin D and/or 25-OH D3 are known, especially regarding bone health, little is known about Vitamin D and its derivatives' effect on phosphorus utilization in the animal.

For example, Tritsch et al. (US 2003/0170324) disclose a feed premix composition of at least 25-OH D3 in an amount between 5% and 50% (wt/wt) dissolved in oil and an antioxidant, an agent encapsulating droplets of 25-OH D3 and oil, and a nutritional additive (e.g., Vitamin D3). The premix may be added to poultry, swine, canine, or feline feed. This composition stabilizes 25-OH D3 against oxidation.

Simoes-Nunes et al. (US 2005/0064018) discloses adding a combination of 25-OH D3 and Vitamin D3 to animal feed. In particular, about 10 μg/kg to about 100 μg/kg of 25-OH D3 and about 200 IU/kg to about 4,000 IU/kg of Vitamin D3 are added to swine feed. This addition improves the ruminant's bone strength.

Stark et al. (U.S. Pat. No. 5,695,794) disclose adding a combination of 25-OH D3 and Vitamin D3 to poultry feed to ameliorate the effects of tibial dyschondroplasia.

Borenstein et al U.S. Pat. No. 5,043,170 discloses the combination of Vitamin D3 and either 1-alpha-hydroxycholecalciferol or 1-alpha, 25-dihydroxycholecalciferol to improve egg strength and leg strength in laying hens and older hens.

While not wishing to be bound by theory, it appears that 25-OH D3 shows an effect on P metabolism in two ways: On one hand it stimulates P absorption from the gastrointestinal tract and on the other hand it improves bioavailability of P into tissues.

For the purposes of the invention, 25-OH D3 is suitably administered as supplement to animal feed. Feed may be supplemented by admixing 25-OH D3, e.g., as a commercial formulation such as available under the Trademark ROVIMIX® Hy•D® 1.25% to regular feed or by first preparing a premix of a feed component and 25-OH D3 and subsequent mixing the premix with other feed components. The feed can be any conventional ruminant feed. The term feed as used herein comprises both solid and liquid feed as well as drinking fluids such as drinking water.

Particularly, 25-OH D3 can be administered to the animal in form of a slow release bolus either alone or with other compounds such as rumen modifiers, ionophores, methane inhibitors etc or as a formulated powder to a premix containing other minerals, vitamins, amino acids and trace elements which premix is added to regular animal feed and thorough mixing to achieve even distribution therein.

Generally, 25-OH D3 is added to the feed in an amount required to administer from about 0.1 μg to about 5.0 μg, especially about 0.5 μg to about 3.0 μg 25-OH D3 per kg body weight of an individual animal per day.

More specifically, in the manufacture of a ruminant feed in accordance with the invention, from about 10 μg/kg to about 1000 μg/kg of 25-OH D3 are suitably added to regular ruminant feed. Alternatively, a feed premix may be prepared on the basis of regular feed components by adding active ingredients to such feed components in higher concentration, e.g., in a concentration of from about 10 mg/kg to about 1000 mg/kg of 25-OH D3. If one kg of such premix is added per 1000 kg of regular feed this would typically meet the individual need of the animal by normal feed consumption.

For dairy cows and beef cattle, the dosage can range from about 10 μg to about 1000 μg per kg cattle feed diet, preferably from about 50 to about 80 μg 25-OH D3 per kg diet. For other ruminants, the amounts are similar, and can be based on the amount of feed given per day. For optimum results, the 25-OH supplementation should be part of a diet which contains an adequate supply of nutrients, generally known to be of benefit to the organism.

The efficiency of the treatment of cattle in accordance with the invention can be seen from the experiments described below.

In an experimental cattle farm, two groups of animals were selected for the following experiment with a low Phosphorous diet.

One group received the control diet which was the standard roughage diet without the supplementation of 25-hydroxy-vitamin D₃. The other group received the experimental diet which was the standard roughage diet supplemented with 2,000 μg 25-hydroxy-vitamin D₃ per head/day

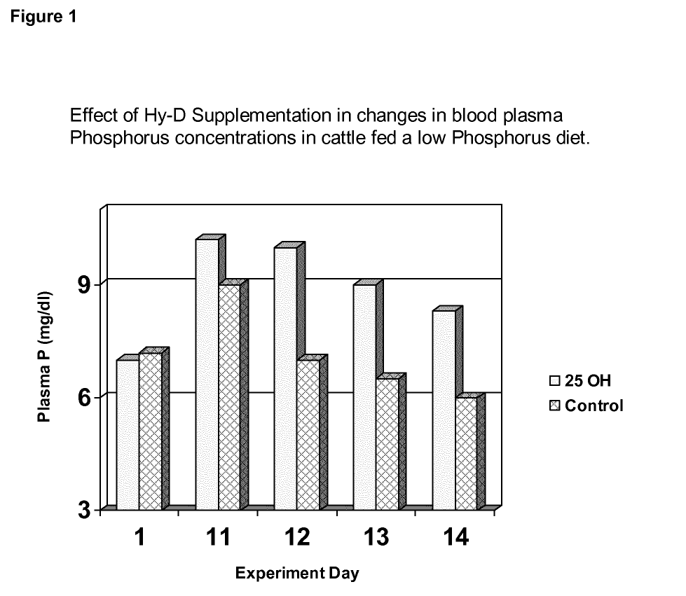

After 1, 11, 12, 13 and 14 days the blood plasma concentration of phosphorous was measured by standard method. The results (FIG. 1) show a significant increase in phosphorous concentration in blood plasma for the group with the experimental diet at least after 10 days.

Additionally the phosphorous concentration in g/day was measured or/calculated in the diet, faeces and urine. The results are summarized in the table below and are consistent with measured plasma concentrations as shown in FIG. 1. The results also show a significant decrease of P excretion by the animal after 25-OH D3 supplementation.

| | | 25OH-D3 | SE | Control | SE | P Value |
|---|---|---|---|---|---|---|
| P (g/day) | Feed | 26.36 | 0.29 | 26.09 | 0.50 | NS |
| | Faeces | 17.21 | 0.44 | 20.38 | 1.00 | P < 0.05 |
| | Urine | 1.12 | 0.27 | 0.85 | 0.18 | NS |
| | Retained | 8.03 | 0.26 | 4.86 | 0.58 | P < 0.05 |

1. A method of promoting phosphorous utilization in a ruminant animal and reducing phosphorous concentration in the excrement of the animal, comprising administering a sufficient amount of 25-hydroxy vitamin D3 to the animal.

2. A method according to claim 1 wherein the ruminant is selected from the group consisting of cattle, goats, sheep and camels.

3. A method according to claim 1 wherein 25-hydroxy-vitamin D₃ is added to the feed in an amount required to administer from about 0.1 μg to about 5.0 μg, especially about 0.5 μg to about 3.0 μg of 25-hydroxy-vitamin D₃ per kg body weight of an individual animal per day.

4. A method according to claim 1 wherein the amount of 25-hydroxy Vitamin D3 administered to the animal is from about 10 μg to about 1000 μg per kg feed.

5. A method according to claim 4 wherein the amount is from about 50 μg to about 80 μg per kg feed.

6. A method according to claim 1 wherein 25-hydroxy-vitamin D₃ is administered as a feed supplement.

7. Use of 25-hydroxy-vitamin D₃ for promoting phosphorous utilization in a ruminant animal and reducing phosphorous concentration in the excrement of the animal.

8. The use according to claim 7 wherein the ruminant is selected from the group consisting of cattle, goats, sheep and camels

9. The use according to claim 7 wherein 25-hydroxy-vitamin D₃ is added to the feed in an amount required to administer from about 0.1 μg to about 5.0 μg, especially about 0.5 μg to about 3.0 μg of 25-hydroxy-vitamin D₃ per kg body weight of an individual animal per day.

10. The use according to claim 7 wherein 25-hydroxy-vitamin D₃ is added to the feed in an amount of about 10 μg to about 1000 μg per kg feed.

11. The use according to claim 10, wherein 25-hydroxy-vitamin D₃ is added to the feed in an amount from about 50 μg to about 80 μg per kg feed.

12. The use according to claim 7 wherein 25-hydroxy-vitamin D₃ is administered as a feed supplement.
